(19) United States
(12) Patent Application Publication
(10) Pub. No.: US 2002/0151481 A1
(43) Pub. Date: Oct. 17, 2002

(54) MMP-2 propeptide for use as antiangiogenic or antitumor agent

(76) Inventor: Lawrence Weissbach, Boston, MA (US)

Correspondence Address: JANIS K. FRASER, PH.D., J.D., FISH & RICHARDSON P.C., 225 Franklin Street, Boston, MA, 02110-2804, US

(21) Appl. No.: 09/833,747

(22) Filed: Apr. 12, 2001

Related U.S. Application Data
(60) Provisional application No. 60/200,115, filed on Apr. 27, 2000.

(51) Int. Cl. A61K 38/08; A61K 38/16; A61K 38/10

(52) U.S. Cl. 514/12; 514/16; 514/15; 514/13; 514/14

(57) ABSTRACT

Methods and compositions for inhibiting growth of a tumor, inhibiting angiogenesis and inhibiting extracellular matrix destruction in a mammal are disclosed. The method includes administering a therapeutically effective amount of a polypeptide that contains the sequence ProArgCysGlyXaaProAsp, wherein Xaa represents Val or Asn (SEQ ID NO:6). Preferably, the polypeptide is 60 to 100 amino acids in length. In some embodiments, the polypeptide is a human MMP-2 propeptide (SEQ ID NO:1) or an MMP-2 propeptide-like polypeptide, i.e., a polypeptide having at least 80% sequence identity with SEQ ID NO:1.

### CROSS-REFERENCE TO RELATED APPLICATIONS

This application claims priority from U.S. Provisional Application Serial No. 60/200,115, filed Apr. 27, 2000.

### TECHNICAL FIELD

This invention relates to molecular biology and oncology.

### BACKGROUND

Matrix metalloproteinases (MMPs), also called matrixins, are involved in embryonic development, morphogenesis, reproduction, tissue resorption and tissue remodeling. They do so through their proteolytic role in the breakdown of extracellular matrix (ECM) (Nagase et al., 1999, J Biol. Chem. 274:21491-21494). This ECM destruction is particularly evident in pathological conditions such as osteoarthritis, rheumatoid arthritis, periodontal disease and atherosclerosis (Skotnicki, 1999, Ann. NY Acad. Sci 878:61-72). MMPs also have been implicated in tumor cell invasion and angiogenesis (Chambers et al., 1997, J. Natl. Cancer Inst. 89:1260-1270; Rosenthal et al., 1998, Cancer Res. 58:5221-5230; Stetler-Stevenson, 1999, J. Clin. Invest. 103:1237-1241). Most MMPs are synthesized as prepro-enzymes and are secreted as inactive pro-MMPs. An N-terminal propeptide of approximately 80 amino acids is cleaved from the human MMP-2 pro-enzyme during conversion of the inactive pro-enzyme to the active enzyme (Massova et al., 1998, FASEB J. 12:1075-1095). The MMP-2 propeptide contains a conserved PRCG(V/N)PD motif (SEQ ID NO:6). The cysteine residue in this motif, sometimes called a “cysteine switch,” ligates the catalytic zinc to maintain the inactivity of pro-MMPs (Van Wart et al., 1990, Proc. Natl. Acad. Sci. USA 87:5578-5582; Becker et al., 1995, Protein Sci. 4:1966-1976). The proteolytic activities of MMPs are controlled, in part through inhibition by endogenous inhibitors such as α-macroglobulins and tissue inhibitors of metalloproteinases (TIMPs).

### SUMMARY

The invention features a method of inhibiting growth of a tumor in a mammal, e.g., a human patient. The method includes identifying a mammal whose body contains a tumor, and administering to the mammal a therapeutically effective amount of a polypeptide that contains the sequence ProArgCysGlyXaaProAsp, wherein Xaa represents Val or Asn (SEQ ID NO:6). The polypeptide can be 60 to 100 amino acids in length. In some embodiments, the polypeptide is a human MMP-2 propeptide (SEQ ID NO:1) or an MMP-2 propeptide-like polypeptide, i.e., a polypeptide having at least 80% sequence identity with SEQ ID NO:1. In some embodiments of the invention, an MMP-2 propeptide-like polypeptide consists of an amino acid sequence differing from SEQ ID NO:1 by 1 to 10 conservative amino acid substitutions. In some embodiments, the polypeptide is fused to an N-terminal polyhistidine tag. Preferably, the MMP-2 propeptide or an MMP-2 propeptide-like polypeptide is administered parenterally. The polypeptide can be administered systemically. Alternatively, it can be administered locally to a tumor site. The therapeutically effective amount can be 1 to 300 mg/kg body weight/day, e.g., 10-30 mg/kg body weight/day.

The invention also features a method of inhibiting angiogenesis in a mammal, e.g., a human patient. The method includes administering to the mammal a therapeutically effective amount of a polypeptide that contains the sequence ProArgCysGlyXaaProAsp, wherein Xaa represents Val or Asn (SEQ ID NO:6). The polypeptide can be 60 to 100 amino acids in length. In some embodiments, the polypeptide is a human MMP-2 propeptide (SEQ ID NO:1) or an MMP-2 propeptide-like polypeptide, i.e., a polypeptide having at least 80% sequence identity with SEQ ID NO:1.

The invention also features a method of inhibiting ECM destruction in a mammal, e.g., a human patient. The method includes administering to the mammal a therapeutically effective amount of a polypeptide that contains the sequence ProArgCysGlyXaaProAsp, wherein Xaa represents Val or Asn (SEQ ID NO:6). The polypeptide can be 60 to 100 amino acids in length. In some embodiments, the polypeptide is a human MMP-2 propeptide (SEQ ID NO:1) or an MMP-2 propeptide-like polypeptide, i.e., a polypeptide having at least 80% sequence identity with SEQ ID NO:1.

The invention also features a pharmaceutical composition. The composition includes a polypeptide that contains the sequence ProArgCysGlyXaaProAsp, wherein Xaa represents Val or Asn (SEQ ID NO:6). The polypeptide can be 60 to 100 amino acids in length. The polypeptide can be a human MMP-2 propeptide (SEQ ID NO:1) or an MMP-2 propeptide-like polypeptide, i.e., a polypeptide having at least 80% sequence identity with SEQ ID NO:1. In some embodiments, an MMP-2 propeptide-like polypeptide consists of an amino acid sequence differing from SEQ ID NO:1 by 1 to 10 conservative amino acid substitutions. In some embodiments, the polypeptide is fused to an N-terminal polyhistidine tag.

Unless otherwise defined, all technical and scientific terms used herein have the same meaning as commonly understood by one of ordinary skill in the art to which this invention belongs. In case of conflict, the present application, including definitions, will control. All publications, patents and other references mentioned herein are incorporated by reference.

Although methods and materials similar or equivalent to those described herein can be used in the practice or testing of the present invention, preferred methods and materials are described below. The materials, methods and examples are illustrative only, and they are not to be construed as limiting the scope or content of the invention in any way. Other features and advantages of the invention will be apparent from the detailed description and from the claims.

### BREIF DESCRIPTION OF THE DRAWINGS

FIG. 1 is a computer printout showing an alignment of amino acids 30-109 of human MMP-2 (SEQ ID NO:1) and amino acids 30-109 of mouse MMP-2 (SEQ ID NO:1). This align comparison was done using the BLASTP 2.0 program, performing an ungapped alignment and using the default parameters. In this alignment, the mouse sequence is 100% identical to the human sequence.

FIG. 2 is a computer printout showing an alignment of amino acids 30-109 of human MMP-2 (SEQ ID NO:1) and amino acids 30-109 of rat MMP-2 (SEQ ID NOs:1 and 7). This align comparison was done using the BLASTP 2.0 program, performing an ungapped alignment and using the default parameters. Two rat sequences were found in the database. In these alignments, one rat sequence (SEQ ID NO:1) is 100% identical, and the other (SEQ ID NO:7) is 98% identical to the human sequence.

FIG. 3 is a computer printout showing an alignment of amino acids 30-109 of human MMP-2 (SEQ ID NO:1) and amino acids 31-109 of rabbit MMP-2 (SEQ ID NO:8). This align comparison was done using the BLASTP 2.0 program, performing an ungapped alignment and using the default parameters. In this alignment, the rabbit sequence is 94% identical to the human sequence.

FIG. 4 is a computer printout showing an alignment of amino acids 30-109 of human MMP-2 (SEQ ID NO:1) and amino acids 27-106 of chicken MMP-2 (SEQ ID NO:10). This align comparison was done using the BLASTP 2.0 program, performing an ungapped alignment and using the default parameters. In this alignment, the chicken sequence is 90% identical to the human sequence.

FIG. 5 is a histogram summarizing the results of experiments conducted to determine the effect of recombinant MMP-2 propeptide on viability of cells of a cultured chondrosarcoma cell line.

FIG. 6 is a histogram summarizing the results of experiments conducted to determine the effect of recombinant MMP-2 propeptide on bovine chondrocytes DNA synthesis.

FIG. 7 is a histogram summarizing the results of experiments conducted to determine the effect of recombinant MMP-2 propeptide on bovine chondrocytes proteoglycan synthesis.

FIG. 8 is a graph showing the effect of recombinant MMP-2 propeptide (1.7 mg/kg) on the growth of human melanoma cell line (H187) in mice. Data are expressed as the mean±standard deviation.

FIG. 9 is a graph showing the effect of recombinant MMP-2 propeptide (3.3 mg/kg) on the growth of human melanoma cell line (H187) in mice. Data are expressed as the mean±standard deviation.

### DETAILED DESCRIPTION

By interfering with tumor-induced neovascularization, polypeptides of the invention exert a powerful repressive effect on tumor growth. This growth inhibition is largely due to a restriction on nutrients and oxygen carried by the vascular system to sustain and nourish tumors. Such restriction on nutrients and oxygen leads to a cytostatic effect on the tumor cells. Proteins and protein fragments that function in maintaining the normal quiescent state of endothelial cells, the key building blocks of blood vessels, are used in the invention to suppress tumor growth and function. These agents can be employed together with conventional anticancer treatments such as surgery, radiation, and chemotherapy. In addition, other pathological conditions characterized by excessive neovascularization can be treated using methods and compositions of the invention. Such other pathological conditions include diabetic retinopathy, rheumatoid arthritis, age-related macular degeneration, and psoriasis.

By interfering with the enzymatic activity of MMPs, e.g., MMP-2, polypeptides of the invention can be employed to reduce unwanted ECM destruction. By virtue of reducing ECM destruction, these polypeptides are useful in treatment of diseases or disorders that involve ECM destruction, e.g., osteoarthritis, rheumatoid arthritis, periodontal disease and atherosclerosis

### Polypeptides

An anti-proliferative, antiangiogenic or ECM destruction-inhibiting polypeptide suitable for use in the methods and compositions of the invention is the 80-amino acid sequence of SEQ ID NO:1. This polypeptide is identical with an N-terminal fragment of human MMP-2, and is known as the MMP-2 propeptide. Other polypeptides useful in the invention are MMP-2-like polypeptides whose amino acid sequence has at least 80% sequence identity with SEQ ID NO:1. In some embodiments, the differences consist of 1, 2, 3, 4, 5, 6, 7, 8, 9, or 10 conservative amino acid substitutions. In other embodiments, the differences consist of additions or deletions of up to 16 amino acids, e.g., deletion of a block of amino acids at the amino terminus or carboxy terminus of the polypeptide defined by SEQ ID NO:1. Anti-proliferative, antiangiogenic or ECM destruction-inhibiting activities of human MMP-2 propeptide or human MMP-2-like propeptides can be assessed, without undue experimentation, by using one or more of the assays described in the Examples below, or by using any other suitable assay. Although an anti-proliferative, antiangiogenic or ECM destruction-inhibiting polypeptide of the invention can contain one or more amino acid sequences in addition to the ProArgCysGlyXaaProAsp (SEQ ID NO:6) motif, human MMP-2 propeptide or human MMP-2-like propeptide, preferably the polypeptide does not contain an MMP catalytic site.

Human MMP-2 propeptide or an MMP-2 propeptide-like polypeptide can be obtained by any suitable method. The MMP-2 propeptide can be produced using conventional recombinant DNA technology, as described in the Examples below. Guidance and information concerning methods and materials for production of polypeptides by recombinant DNA technology can be found in numerous treatises and reference manuals, e.g., Sambrook et al., 1989, Molecular Cloning—A Laboratory Manual, 2^{nd} Ed., Cold Spring Harbor Press; Ausubel et al. (eds.), 1994, Current Protocols in Molecular Biology, John Wiley & Sons, Inc.; Innis et al. (eds.), 1990, PCR Protocols, Academic Press.

Alternatively, the MMP-2 propeptide or modification thereof can be obtained directly by chemical synthesis, e.g., using a commercial peptide synthesizer. Methods and materials for chemical synthesis of polypeptides are well known in the art. See, e.g., R. B. Merrifield, 1963, “Solid Phase Peptide Synthesis,” J. Am. Chem. Soc. 83:2149-2154.

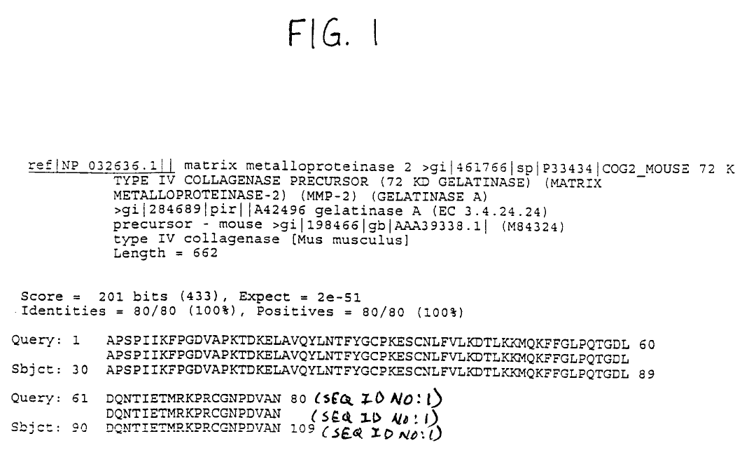

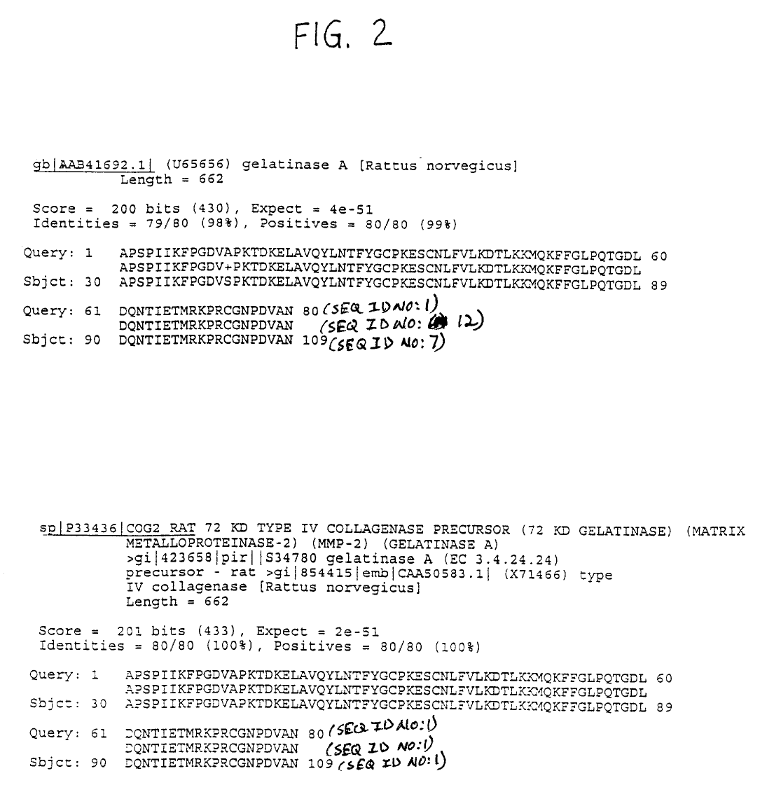

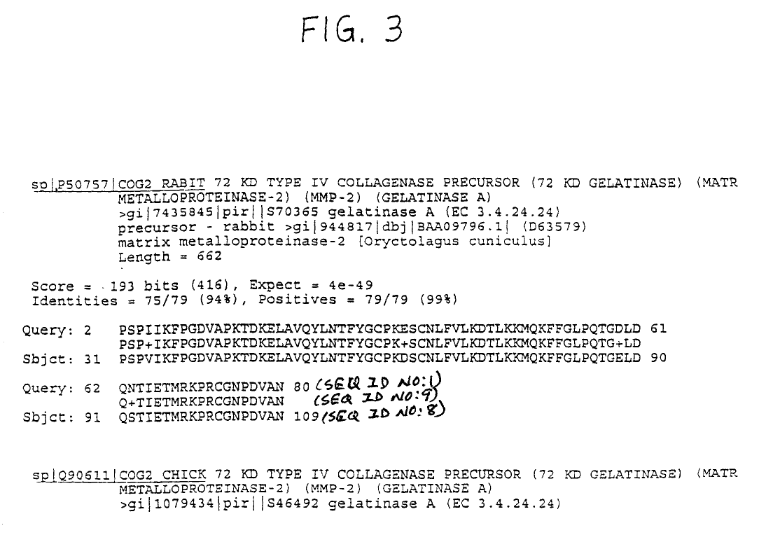

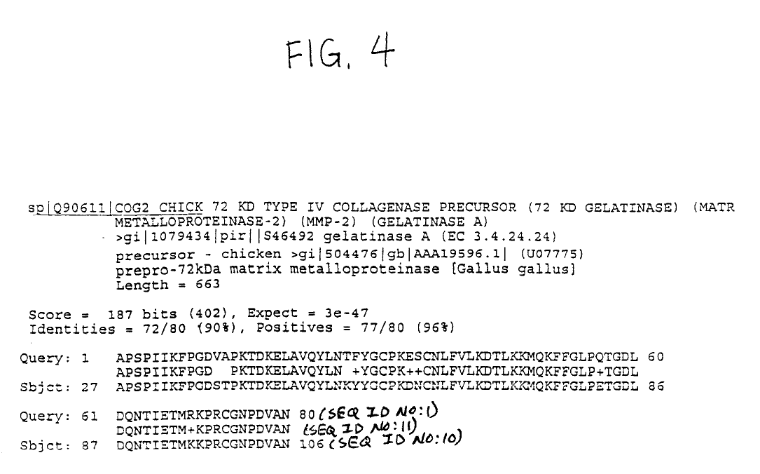

Useful modifications of human MMP-2 propeptide are exemplified by MMP-2 propeptides from other species. The MMP-2 propeptide from mouse (Mus musculus) is 100% identical with the human MMP-2 propeptide (FIG. 1). The MMP-2 propeptide from rat (Rattus norvegious) is 100% or 98% identical with the human MMP-2 propeptide (FIG. 2). The MMP-2 propeptide from rabbit (Oryctolagus cuniculus) is 94% identical with the human MMP-2 propeptide (FIG. 3). The MMP-2 propeptide from chicken (Gallus gallus) is 90% identical with the human MMP-2 propeptide (FIG. 4).

The determination of percent identity between two amino acid sequences is accomplished using the BLAST 2.0 program, which is available to the public on the Internet. See, e.g., the website for the National Center for Biotechnology Information (NCBI). Sequence comparison is performed using an ungapped alignment and using the default parameters (Blossom 62 matrix, gap existence cost of 11, per residue gap cost of 1, and a lambda ratio of 0.85). The mathematical algorithm used in BLAST programs is described in Altschul et al., 1997, Nucleic Acids Research 25:3389-3402.

An MMP-2 propeptide or modification thereof can be administered according to the invention by any suitable method. Preferably the polypeptide is administered parenterally, to avoid digestion in the stomach. Parenteral administration can be systemic, e.g., by an intravenous route. In some embodiments, the polypeptide is administered locally, e.g., by direct injection into a tumor.

### Polypeptide Dosage, Formulation and Administration

MMP-2 and other MMPs are secreted into intracellular spaces such as extracellular matrices. Therefore, in the practice of the invention, the MMP-2 propeptide or MMP-2 propeptide-like polypeptide need not cross cytoplasmic membranes or otherwise enter into cells.

The dosage of the MMP-2 propeptide or MMP-2 propeptide-like polypeptide can be 1-300 mg/kg body wt/day, e.g., 10-30 mg/kg body wt/day. Optimization of dosage will depend on factors such as location, size, and type of tumor being treated, route of administration, age and condition of the patient. Such optimization is within ordinary skill in the art.

MMP-2 propeptide or an MMP-2 propeptide-like polypeptide can be formulated into pharmaceutical compositions by admixture with pharmaceutically acceptable nontoxic excipients and carriers. Such compositions can be prepared for use in parenteral administration, particularly in the form of liquid solutions or suspensions; for oral administration, particularly in the form of liquid, tablets or capsules; or intranasally, particularly in the form of powders, nasal drops, or aerosols.

The composition can be administered conveniently in unit dosage form and can be prepared by any of the methods known in the art. Such methods are described, for example, in Remington's Pharmaceutical Sciences (Mack Pub. Co., Easton, Pa., 1980).

Liquid dosage forms for oral administration include pharmaceutically acceptable emulsions, microemulsions, solutions, suspensions, syrups and elixirs. In addition to the active compound, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate. Other additives include (a) fillers or extenders such as starches, lactose, sucrose, glucose, mannitol, and silicic acid; (b) binders such as carboxymethylcellulose, alginates, gelatin, polyvinylpyrrolidinone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents such as cetyl alcohol and glycerol monostearate; (h) absorbents such as kaolin and bentonite clay; and (i) lubricants such as talc, calcium stearate, magnesium stearate, solid polyethylene glycols, sodium lauryl sulfate; and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. In solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

### EXAMPLE

Recombinant Production of MMP-2 Propeptide. The human MMP-2 propeptide was cloned and expressed in a bacterial system as a recombinant fusion protein having an N-terminal polyhistidine tag. The recombinant human MMP-2 propeptide (with polyhistidine tag) consisted of the amino acid sequence MRGSHHHHHH GSAPSPIIKF PGDVAPKTDK ELAVQYLNTF YGCPKESCNL FVLKDTLKKM QKFFGLPQTG DLDQNTIETM RKPRCGNPDV AN (SEQ ID NO:2). The recombinant polypeptide consisting of the amino acid sequence of SEQ ID NO:2 was encoded by the nucleotide sequence

ATGAGAGGATCGCATCACCATCACCATCACGGATCCGCGCCGTCGCCCAT
CATCAAGTTCCCCGGCGATGTCGCCCCCAAAACGGACAAAGAGTTGGCAG
TGCAATACCTGAACACCTTCTATGGCTGCCCCAAGGAGAGCTGCAACCTG
TTTGTGCTGAAGGACACACTAAAGAAGATGCAGAAGTTCTTTGGACTGCC
CCAGACAGGTGATCTTGACCAGAATACCATCGAGACCATGCGGAAGCCAC
GCTGCGGCAACCCAGATGTGGCCAAC
(SEQ ID NO: 3).

The human MMP-2 propeptide was amplified from total liver DNA using RT-PCR as described in Weissbach et al., 1992, Biochem. Biophys. Res. Commun. 186:1108-1114. The oligonucleotide primers contained engineered restriction enzyme recognition sites, and the MMP-2 RT-PCR product was directionally cloned into a bacterial expression plasmid (pQE-30, Qiagen, Valencia, Calif.) using BamHI and KpnI. The sense primer consisted of the sequence TTTAAAGGATCCGCGCCGTCGCCCATCATC (SEQ ID NO:4). The antisense primer consisted of the sequence TCCCAGGGTACCCTAGTTGGCCACATCTGGGTT (SEQ ID NO:5).

Cloning into pQE-30 with a BamHI/KpnI-directed DNA insertion also resulted in an identical 12-amino acid N-terminal tag preceding the MMP-2 propeptide sequence of 80 amino acids. The complete fusion protein of SEQ ID NO:2 has 92 amino acids, a calculated molecular weight of 10,333 Da and an isoelectric point of 8.8.

Purification of Recombinant Polypeptide. The recombinant MMP-2 propeptide with polyhistidine tag was purified from bacterial host cells essentially according to the procedure published in Lewis et al., 1999, Eur. J. Biochem. 259:618-625. Briefly, the polypeptide was solubilized in urea (8 M), isolated via nickel affinity chromatography, and eluted with imidazole. The eluate was then extensively dialyzed against phosphate-buffered saline (PBS). The homogeneity of the polyhistidine-tagged MMP-2 propeptide preparation was confirmed by sodium dodecyl sulfate polyacrylamide gel electrophoresis (SDS-PAGE).

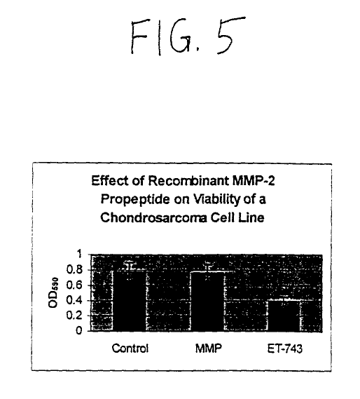

Animal Cell Experiments. The effects of the MMP-2 propeptide on viability and metabolism of cultured animal cells was observed. A human chondrosarcoma cell line was cultured in both the presence and absence of 50 μg/ml recombinant MMP-2 propeptide (serum-free). Cultures were observed over a 48 hours period. Cell viability was monitored via crystal violet nuclear staining and absorption spectroscopy (OD₅₉₀). Absorbance is proportional to cell number (Gillies, 1986, Anal. Biochem. 159:109-113). Cells exposed to MMP-2 propeptide showed no decrease in cell number when compared to control cells (FIG. 5). The compound ET-743 (a chemotherapeutic agent being evaluated in clinical trials (Izbicka et al., 1998, Ann. Oncol. 9:981-987)) was observed to be cytotoxic, as evidenced by a 50% decrease in viable cell number (FIG. 5). That MMP-2 had no effect on the cultured cancer cell line was not unexpected since the effects of angiogenesis inhibitors (such as angiostatin and endostatin) are believed to be restricted to mature endothelial cells.

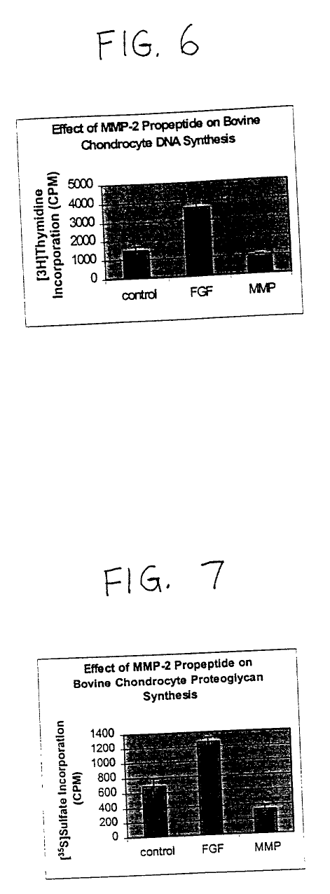

The effect of recombinant MMP-2 propeptide on primary cultures of calf articular chondrocytes was investigated. The effect of the propeptide on both [³H]thymidine incorporation into DNA and [³⁵S]sulfate incorporation into proteoglycans was measured essentially as described in Bonassar et al., 1997, Exp. Cell Res. 234:1-6. Briefly, chondrocytes were isolated from radiocarpal joints of 1 to 2-week old calves by preparing 2-3 mm² slices, transferring to serum-free medium containing 0.08% collagenase, and incubating overnight at 37° C. in a spinner bottle. Released cells were filtered through nylon mesh and washed to remove undigested tissue and collagenase. Cells were plated in serum-containing medium and incubated for 2-4 days. Following the 2-4 day period, the serum-containing medium was replaced with serum-free medium and the cells were allowed to incubate for an additional 48 hours. [³⁵S]sulfate or [³H]thymidine was added to the serum-free medium for the final 16 hours of the incubation period. Cell monolayers were then processed for quantification of radiolabel incorporated into macromolecules. At a concentration of 50 μg/ml, recombinant MMP-2 propeptide suppressed DNA synthesis by 38% (FIG. 6). Proteoglycan synthesis was inhibited by about 50% (FIG. 7). These results demonstrate the antiproliferative or antiangiogenic activities of MMP-2.

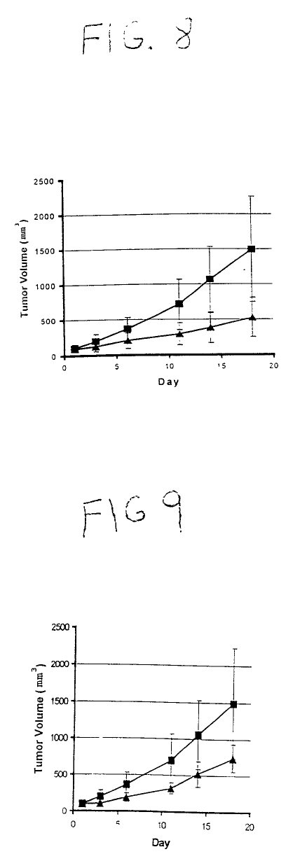

The effects of recombinant MMP-2 propeptide on tumor growth were investigated in vivo. Xenografts consisting of a human melanoma cell line (H187) were implanted subcutaneously into athymic nude mice (CD1 nu/nu), and allowed to grow to a size of about 100 mm³. The mice were then randomized into three groups. Each of the three groups of mice were treated with one of the following: phosphate-buffered saline (control), doses of 1.7 mg recombinant MMP-2 propeptide/kg mouse weight, or doses of 3.3 mg MMP-2 propeptide/kg mouse weight. Doses were administered intraperitoneally once per day for nine consecutive days. Following the nine-day period, tumor volumes in all mice were measured. The recombinant MMP-2 propeptide was shown to inhibit tumor growth at both doses (FIGS. 8 and 9). The percent inhibition was greater for the lower dose than the higher dose (63% and 50% respectively, at day 14). The data were statistically significant (p=0.0005 and 0.002 for the 1.7 and 3.3 mg/kg doses, respectively, based on ANOVA two-factor variance analysis). These data demonstrate that MMP-2 propeptide can interfere with the formation of an active MMP-2 species, resulting in the disruption of angiogenesis and/or tumor cell proliferation.

A number of embodiments of the invention have been described. Nevertheless, it will be understood that various modifications may be made without departing from the spirit and scope of the invention. Accordingly, other embodiments are within the scope of the following claims.

What is claimed is:

1. A method of inhibiting growth of a tumor in a mammal, comprising identifying a mammal whose body comprises a tumor, and administering to the mammal a therapeutically effective amount of a polypeptide that comprises the sequence ProArgCysGlyXaaProAsp, wherein Xaa represents Val or Asn (SEQ ID NO:6).

2. The method of claim 1, wherein the polypeptide is 60 to 100 amino acids in length.

3. A method of inhibiting growth of a tumor in a mammal, comprising identifying a mammal whose body comprises a tumor, and administering to the mammal a therapeutically effective amount of a polypeptide having at least 80% sequence identity with SEQ ID NO:1.

4. The method of claim 3, wherein the polypeptide consists of a sequence differing from SEQ ID NO:1 by 1 to 10 conservative amino acid substitutions.

5. The method of claim 3, wherein the amino acid sequence of the polypeptide consists of SEQ ID NO:1.

6. The method of claim 3, wherein the polypeptide is fused to an N-terminal polyhistidine tag.

7. The method of claim 3, wherein the polypeptide is administered parenterally.

8. The method of claim 7, wherein the polypeptide is administered systemically.

9. The method of claim 7, wherein the polypeptide is administered locally to the tumor site.

10. The method of claim 3, wherein the therapeutically effective amount is 1 to 300 mg/kg body weight/day.

11. The method of claim 10, wherein the therapeutically effective amount is 10-30 mg/kg body weight/day.

12. A method of inhibiting angiogenesis in a mammal, comprising administering to the mammal a therapeutically effective amount of a polypeptide that comprises the sequence ProArgCysGlyXaaProAsp, wherein Xaa represents Val or Asn (SEQ ID NO:6).

13. The method of claim 12, wherein the polypeptide is 60 to 100 amino acids in length.

14. A method of inhibiting angiogenesis in a mammal, comprising administering to the mammal a therapeutically effective amount of a polypeptide having at least 80% sequence identity with SEQ ID NO:1.

15. The method of claim 14, wherein the polypeptide consists of a sequence differing from SEQ ID NO:1 by 1 to 10 conservative amino acid substitutions.

16. The method of claim 14, wherein the amino acid sequence of the polypeptide consists of SEQ ID NO:1.

17. The method of claim 14, wherein the polypeptide is fused to an N-terminal polyhistidine tag.

18. The method of claim 14, wherein the polypeptide is administered parenterally.

19. The method of claim 14, wherein the polypeptide is administered systemically.

20. The method of claim 18, wherein the polypeptide is administered locally to the tumor site.

21. The method of claim 14, wherein the therapeutically effective amount is 1 to 300 mg/kg body weight.

22. The method of claim 21, wherein the therapeutically effective amount is 10 to 30 mg/kg body weight.

23. A method of inhibiting extracellular matrix destruction in a mammal, comprising administering to the mammal a therapeutically effective amount of a polypeptide that comprises the sequence ProArgCysGlyXaaProAsp, wherein Xaa represents Val or Asn (SEQ ID NO:6).

24. The method of claim 23, wherein the polypeptide is 60 to 100 amino acids in length.

25. A method of inhibiting extracellular matrix destruction in a mammal, comprising administering to the mammal a therapeutically effective amount of a polypeptide having at least 80% sequence identity with SEQ ID NO:1.

26. The method of claim 25, wherein the polypeptide consists of a sequence differing from SEQ ID NO:1 by 1 to 10 conservative amino acid substitutions.

27. The method of claim 25, wherein the amino acid sequence of the polypeptide consists of SEQ ID NO:1.

28. The method of claim 25, wherein the polypeptide is fused to an N-terminal polyhistidine tag.

29. The method of claim 25, wherein the polypeptide is administered parenterally.

30. The method of claim 25, wherein the polypeptide is administered systemically.

31. The method of claim 29, wherein the polypeptide is administered locally to the tumor site.

32. The method of claim 25, wherein the therapeutically effective amount is 1 to 300 mg/kg body weight.

33. The method of claim 32, wherein the therapeutically effective amount is 10 to 30 mg/kg body weight.

34. A pharmaceutical composition comprising a polypeptide that comprises the sequence ProArgCysGlyXaaProAsp, wherein Xaa represents Val or Asn (SEQ ID NO:6).

35. A pharmaceutical composition comprising a polypeptide having at least 80% sequence identity with SEQ ID NO:1.

36. The composition of claim 35, wherein the polypeptide consists of an amino acid sequence differing from SEQ ID NO:1 by 1 to 10 conservative amino acid substitutions.

37. The composition of claim 35, wherein the amino acid sequence of the polypeptide consists of the amino acid sequence of SEQ ID NO:1.
